(19) United States
(12) Patent Application Publication
(10) Pub. No.: US 2009/0170949 A1
(43) Pub. Date: Jul. 2, 2009

(54) Method To Control Body Weight

(76) Inventors: Soraya Shirazi-Beechey, Wirral (GB); Dirk Iserentant, Wijgmaal (BE)

Correspondence Address: NIXON & VANDERHYE, PC, 901 NORTH GLEBE ROAD, 11TH FLOOR, ARLINGTON, VA, 22203, US

(21) Appl. No.: 12/087,597

(22) PCT Filed: Jan. 23, 2007
(86) PCT No.: PCT/EP2007/050647

§ 371 Date: Jul. 10, 2008

(30) Foreign Application Priority Data
Jan. 25, 2006 (EP) 06100804.1

(51) Int. Cl. A61K 31/192; A61P 3/04; A61P 3/10

(52) U.S. Cl. 514/570

(57) ABSTRACT

The present invention relates to a method to treat obesity and/or a method to lose or control body weight. More specifically, the invention relates to the use of a compound inhibiting the sweet taste perception, preferably by inactivating the T1R3 receptor, for the preparation of a medicament to treat or prevent obesity and/or to treat or prevent diabetes and/or to lose or control body weight.

The present invention relates to a method to treat obesity and/or a method to lose or control body weight. More specifically, the invention relates to the use of a compound inhibiting the sweet taste perception, preferably by inactivating the T1R3 receptor, for the preparation of a medicament to treat or prevent obesity and/or to treat or prevent diabetes and/or to lose or control body weight.

Obesity and diabetes are becoming major problems in the western world, largely developed because of a fat and sugar overconsumption. In recent years, several low fat and low sugar foods have been developed, to restrict the high energy intake of the consumers, without the need for a drastic change in their feeding pattern. However, in spite of the overwhelming presence of so called light food in the market, the epidemic growth of overweight, obesity and diabetes has not been reduced.

There are indeed conflicting results between the use of light foods and drinks and the loss of body weight. Bellisle et al. (2001) noticed in a longitudinal study of 8 years that regular and high consumers of low sugar products but taking artificial sweeteners were not losing, but gaining weight, body mass index and waist and hip size. A similar effect was noticed in both male and female dogs, fed with a diet comprising sucralose as artificial sweetener. For various concentrations of sucralose used, the dogs consuming the artificial sweetener were gaining more rapidly body weight and reached a higher final body weight level (Goldschmidt, 2000). These conflicting results might be explained through the mechanism of nutrient sensing in the intestine, and the subsequent enhancement in the expression and/or level of intestinal sugar transporters. Indeed, using both in vivo and in vitro models it has been shown that the activity and the expression of Na⁺/glucose cotransporter SGLT1 is directly regulated by the luminal (medium) monosaccharides, and that the metabolism of glucose is not required for the glucose induction of SGLT1 (Ferraris and Diamond, 1989; Solberg and Diamond, 1987; Lescale-Matys et al., 1993; Shirazi-Beechey, 1996; Dyer et al., 1997). Furthermore a membrane impermeable glucose analogue, when introduced into the lumen of the intestine, also stimulates SGLT1 expression and abundance, implying that a glucose sensor expressed on the luminal membrane of the intestinal cells is involved in sensing the luminal sugar (Dyer et al., 2003).

The only knowledge of sugar sensing in the mammalian gastrointestinal tract is from taste transduction mechanisms. Taste cells in the taste buds of the tongue epithelium have mechanisms that can distinguish chemical compounds, such as sugars, having potential nutritional value. It has been shown that transduction of sweet-tasting compounds involves activation of G-protein coupled receptor (GPCR) on the apical surface of taste receptor cells.

Recent studies indicate that the members of the taste T1R receptor family (T1R2/T1R3) and gustducin, a taste-specific transducin-like G-protein α subunit, are involved in transduction of sugars in the tongue.

Recently we were able to demonstrate that taste receptors, T1R1-3, which were thought to be limited in expression to the tongue, are expressed in the small intestine. Furthermore we demonstrate that the receptors along with Gα_{gust} are expressed luminally, and mainly in the proximal region of the small intestine.

These GPCRs are involved in sensing dietary glucose, initiating a signaling pathway which ultimately leads to an enhancement in SGLT1 expression, upon activation of the receptor. Surprisingly we found that the activation of the receptor and the consequent enhancement in SGLT1 expression is not only caused by glucose, but also by artificial sweeteners such as sucralose. As an unexpected consequence, addition of an artificial sweetener to a low carbohydrate diet will lead to increased SGLT1 expression, resulting in a more efficient uptake of the remaining sugar, and hence a better food conversion. Therefore, in agreement with the observations by Goldschmidt (2000) and Bellisle et al (2001), but contrary to the generally accepted believe that a low carbohydrate diet with artificial sweeteners will result in a body weight loss, the addition of an artificial sweetener to a low carbohydrate diet will increase the intestinal adsorption of dietary sugars, resulting in a body weight gain.

Even more surprisingly, we found that the addition of a compound blocking the sweet taste, such as lactisole, is resulting in a body weight loss. This effect is obtained even when the compound is encapsulated and/or coated to avoid contact with the taste receptor of the mouth. Therefore, such compounds can be used to lose or control body weight, or to treat obesitas. This is especially unexpected as Lactisole [sodium 2-(4-methoxyphenoxy) propanoate] is a food additive with GRAS status, and has extensively be tested, whereby it is believed that Lactisole has no effect on glucose metabolism or insulin, C-peptide of glucagon secretion (WHO study). Although Hill and Wood (1986, as cited in WHO food additives 50) found a reduction in body-weight gain when high lactisole concentrations were added to the diet, they considered the results as statistically insignificant. However, this study has been carried out in rats, and recent research has indicated that rats are rather insensitive to lactisole (Winnig et al, 2005). Therefore, the effect of lactisole on body weight should be tested in other animals than rodents.

A first aspect of the invention is the use of a compound inhibiting sweet taste for the preparation of a medicament to treat or prevent obesity and/or to treat or prevent diabetes and/or to lose or control body weight. Sweet taste inhibitors are known to the person skilled in the art, and have, as a non-limiting example been disclosed in the UK patent applications 2157148 and 2180534, in the U.S. Pat. Nos. 4,544,565, 4,567,053 and 4,642,240 and in the patent applications EP0351973 and WO9118523. Preferably, said inhibitor has the structure

X^{+ -}OOC—(CO)ₘ(CHR)ₙ—(O)_{q}-Φ(R′)ₚ

wherein m represents 0 or 1 and when m is 0, n is 1, 2 or 3 and p represent 1, 2, 3 or 4 and when m is 1, n is 1 or 2 and p is 0, 1, 2, 3 or 4; q represents 0 or 1; R represents H or lower alkyl; R′ represents a lower alkoxy group, a phenoxy group a lower alkyl group or a trifluoromethyl group, or two R′ substituents taken together represent an aliphatic chain linked to the phenyl group (Φ) at two positions, either directly of via an oxa group, or one R′ substituent represent a hydroxyl group while at least one other R′ substituent represents an alkoxy group; X⁺ represents a physiological acceptable cation such as H⁺ or Na⁺. Preferably, said inhibitor is a propanoic acid derivative, a propionic acid derivative, a methylpropionic acid derivative, a dimethylpropionic acid derivative or an acceptable salt thereof. Even more preferably, said inhibitor is 2-(4-methoxyphenoxy)propanoic acid, most preferably the sodium salt of it.

Preferably, said compound is processed to avoid the inactivation of the taste receptor in the mouth during the treatment. Avoiding contact with the taste receptor in the mouth is important, because otherwise the pills would have a negative influence on the taste of foodstuffs, as the maximal effect of the pills is expected when given shortly before food intake. Methods of processing are known to the person skilled in the art and include, but are not limited to encapsulation in gelatin capsules or equivalent materials, or coating of the tablets with materials such as Eudragit®. Even more preferably, said encapsulation is protecting said compound against the acidity in the stomach, whereby the compound is released in the intestine. This can be realized by methods such as enteric coating. Methods for enteric coating are known to the person skilled in the art and include, but are not limited to polymers such as Eudragit® and Instacoat™

Blocking the sugar transport at the level of the taste receptor, rather than at the level of the sugar transporter has as advantage that a basal level of sugar transport is remaining, and by this avoiding possible problems that may be caused by a complete sugar starvation.

Another aspect of the invention is the use of the taste receptor (T1R2-T1R3) or one of its receptor subunits for the screening of compounds useful to treat obesity and/or diabetes. Indeed, as blocking the sweet taste receptor results in a lower activity of the sugar transporter SGLT1, compounds influencing the activity of the sweet taste receptor are interesting as possible therapeutic compounds. Testing the activity of the compound can be done in vivo, by adding the compound to high sugar diet and screening for compounds that downregulate the SGLT1 expression in the intestine, or it may be done in vitro, by using epithelial cells expressing the sweet taste receptor, and using a reporter gene functionally linked to the SGLT1 promoter. A reporter gene can be any suitable reporter, such as, as a non limiting example, a GFP gene or a luciferase gene, or it can be the SGLT1 protein itself. Alternatively the umami (T1R1-T1R3) receptors may be used for screening. Indeed, as both the sweet taste and umami receptor share one subunit, inhibition of the umami receptor may be due to an inhibition of the T1R3 subunit.

### BRIEF DESCRIPTION OF THE FIGURES

FIG. 1: The effect of dietary carbohydrate level on SGLT1 expression in the small intestine of wild-type, α-gustducin and T1R3 KO mice. a, Real-time PCR data of SGLT1 mRNA levels, normalised to β-actin, in wild-type mouse proximal, mid, and distal intestine maintained on low carbohydrate (LC), high carbohydrate (HC) and LC plus sucralose diets for 2 weeks. Data are mean±S.E.M. (n=4). b, Representative western blot analysis of luminal membrane vesicles isolated from the proximal intestine of wild-type mice. c, Real-time PCR data of SGLT1 expression in the proximal intestine of wild-type and KO mice in response to diet. Data are mean±S.E.M. (n=4).

FIG. 2: Effect of sucralose on body weight gain of mice. Average body weight gain of mice, put on a low carbohydrate diet with or without sucralose.

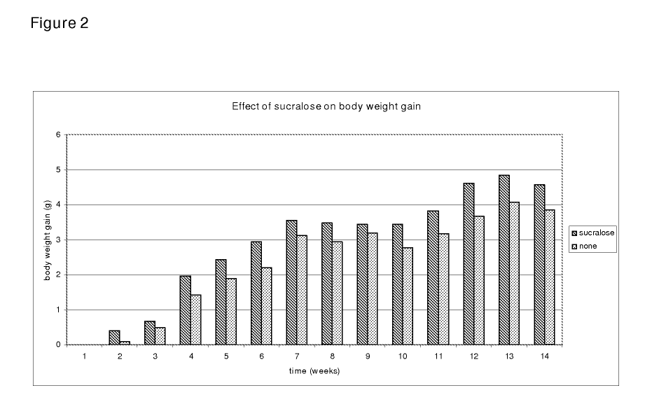

### EXAMPLES

### Materials and Methods to the Examples

### Animals and Tissue Collection.

Male CD-1 and C57BL/6 mice, six weeks old, from Charles River Laboratories were used. The α-gustducin knock out mouse was described by Wong et al. (1996); the T1R3 knock out mouse was described by Damak et al. 2003).

High and low carbohydrate diets were resp. TestDiet® 5810 and TestDiet® 5787-9. For the sucralose test, the low carbohydrate diet was supplemented with sucralose (1,6-dichloro-1,6-dideoxy-beta-D-fructofuranosyl-4-chloro-4-deoxy-alpha-galactopyranoside) at 2 mM.

Animals were killed by concussion followed by cervical dislocation. The entire small intestine was removed and flushed with ice-cold 0.9% NaCl, opened longitudinally, rinsed in saline and mucous removed by blotting. The small intestine was then divided into proximal, mid and distal sections and the mucosa removed by scraping. Mucosal scrapings were frozen immediately in liquid nitrogen and stored at −80° C. until use.

### Real-Time PCR.

Using the Primer Express software program (Applied Biosystems) PCR primers and probes (FAM/TAMRA labeled) for the amplification of T1R1, T1R2, T1R3, Gα_{gust}, and the Na⁺/glucose co-transporter (SGLT1), along with β-actin (JOE/TAMRA labeled) were designed. Primers and probes were purchased from Eurogentec, along with 18S ribosomal RNA controls.

cDNA was synthesized from either total RNA or mRNA using Supercript III reverse transcriptase (Invitrogen) and either oligo(dT)₁₂₋₁₈ or random primers, cleaned up using the Machery-Nagel Nucleospin extract kit and 50 ng of cDNA used per reaction.

For Real-Time PCR reactions the enzyme was activated by heating at 95° C. for 2 min. A two-step PCR procedure was used, 15 s at 95° C. and 60 s at 60° C. for 45 cycles in a PCR mix containing 5 μl of cDNA template, 1× Jumpstart qPCR master mix (Sigma-Aldrich), 900 nM of each primer and 250 nM probe in a total volume of 25 μl. Where multiplex reactions were performed the β-actin primers were primer limiting and used at 600 nM. All reactions were performed in a RotorGene 3000 (Corbett Research).

### Western Blotting.

Brush-border membrane vesicles were isolated from intestinal mucosal scrapings and isolated cells by the cation precipitation, differential centrifugation technique described previously (Shirazi-Beechey et al. 1990). Membrane proteins were denatured in SDS-PAGE sample buffer (20 mM Tris/HCl, pH 6.8, 6% SDS, 4% 2-mercaptoethanol and 10% glycerol) by heating at 95° C. for 4 min and were separated on 8% polyacrylamide gels and electrotransferred to PVDF membranes. Membranes were blocked by incubation in TTBS plus 5% non-fat milk for 60 min. Membranes were incubated for 60 min with antisera to SGLT1, T1R2 (Santa-Cruz), T1R3 (AbCam), Gα_{gust} (Santa-Cruz), villin (The Binding Site), and β-actin (Sigma-Aldrich) in TTBS containing 0.5% non-fat milk. Immunoreactive bands were visualized by using horseradish peroxidase-conjugated secondary antibodies and enhanced chemiluminescence (Amersham Biosciences). Scanning densitometry was performed using Phoretix 1D (Non-Linear Dynamics

### Example 1

SGLT1 is induced by the artificial sweetener sucralose, by means of the T1R3/α-gustducin pathway

To investigate any direct links between T1Rs, α-gustducin, and SGLT1 expression, we performed dietary trials on T1R3^{-/-} and α-gustducin^{-/-} knock-out mice.

Firstly, groups of wild-type and T1R3 and α-gustducin “knock-out” (KO) mice were placed on standard diets with the same carbohydrate composition for two weeks. After this time the mice were killed and the small intestine removed, divided into proximal, mid and distal regions, and SGLT1 expression at the levels of mRNA and protein was measured. The rates of glucose transport were also determined in brush-border membrane vesicles isolated from the tissues.

There were no differences in the levels of SGLT1 mRNA, SGLT1 protein and glucose transport in the intestine of wild-type and KO mice. Therefore all animals had the capacity to absorb dietary sugars. This was evident since neither groups showed any signs of intestinal malabsorption. The data indicate that there is a constitutive pathway, independent of the luminal sensor, which maintains basal expression of SGLT1.

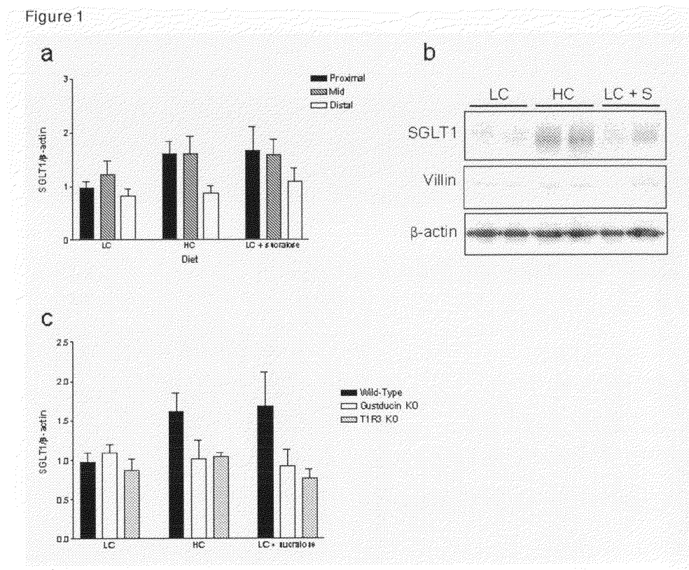

Second, groups of wild-type and T1R3 and α-gustducin KO mice were placed on each of three iso-caloric diets a) low carbohydrate, b) high carbohydrate, and c) low carbohydrate+artificial sweetener (sucralose), for two weeks. After this time the mice were killed and the small intestines were removed, divided into proximal, mid and distal regions, and SGLT1 expression, at protein and mRNA levels, were measured in each. The results are shown in FIG. 1.

FIG. 1A shows the changes in SGLT1 mRNA levels, measured by qPCR in wild-type mice. SGLT1 mRNA is increased 30-70% in the proximal and mid intestinal regions in response to both the high carbohydrate diet and the addition of sucralose to the low carbohydrate diet. Increased SGLT1 expression in mice in response to an increase in dietary carbohydrate has been reported previously, and is a well-established phenomenon. The increase in SGLT1 expression in response to sucralose is a novel finding. Sucralose is marketed as a compound that has no physiological effect on the body other than a sweet taste. It is reported to be non-hydrolyzed, non-transported and non-metabolized within the mammalian small intestine. SGLT1 protein expression is also increased in response to both high carbohydrate and low carbohydrate+sucralose diets (FIG. 1B) in wild-type animals.

In contrast to the wild type situation, there was no increase in SGLT1 mRNA and protein in response to high carbohydrate and low-carbohydrate+sucralose diets in both T1R3 and α-gustducin KO animals (FIG. 1C) proving that both T1R3 and α-gustducin are required for this response as key components of the intestinal sugar-sensor. This novel finding supports our proposition that the taste receptor T1R3 and the G-protein α-gustducin are constituents of the intestinal glucose sensing mechanism which ultimately results in the modulation of SGLT1 expression and the capacity of the small intestine to absorb sugars.

### Example 2

induction of weight increase in mice by the use of an artificial sweetener Two groups of mice (C57BL/6) were fed ad libitum with a low carbohydrate diet (1.9% remaining carbohydrate, Purina), with or without 0.3% sucralose (Tate and Lyle). Food consumption and body weight was followed for a period of 12 weeks.

The body weight gain was higher for the sucralose mice than for the control group. Although the food intake of the sucralose group was slightly higher (7.5%), the average increase in body weight gain (42%) cannot simply be explained by the increase of food intake, and is due to a more efficient food uptake. The difference is specially pronounced at the start of the diet.

### Example 3

### Effect of Coated Lactisole on Metabolic Syndrome in Marmosets

### Preparation of the Pills

5 mm diameter coated pill were made, comprising 20 mg tablettose, 26.5 mg Avicell PH 102, 2.5 mg Crospovidone and 1 mg Mg-stearate for the placebo, and 35 mg sodium 2-(4-methoxyphenoxy)propionate (Endeavour speciality chemicals), 11.5 mg Avicel PH 102, 2.5 mg Crospovidone and 1 mg Mg-stearate for the Lactisole pills. Pills were coated in a fluidized bed (GCPG1, Glatt), at a spray rate of 4 g/min, atomic pressure 1.5 bar, inlet air temperature 36° C., product temperature 31° C. at maximal air velocity. The composition of the coating solution was 11.4% Eudragit® EPO(Röhm Pharma), 1.14% Sodium lauryl sulphate (α-pharma), 4% Mg-stearate (α-pharma), 1.72% stearic acid (α-pharma) and water ad 100%.

### Animals and Feeding Tests

Adult male and female common marmoset monkeys (Callithrix jacchus) were from the breeding colony of the German Primate Center (DPZ), Göttingen, Germany. Animals were housed in pairs in air-conditioned facilities on a 12 hr/12: HR light/dark cycle.

Animals were fed two times a day: mash feeding, containing 15 g of test diet in the morning, and 60 g of test diet in the afternoon (value per animal). Each time before feeding, the animals received two pills (either placebo or lactisole) in nutrical gel.

Three groups of marmosets were compared: 11 obese animals treated with lactisole, 11 obese animals receiving placebo and 12 lean control animals receiving placebo. The experiment is carried out for 10 weeks, and bodyweight, glycated hemoglobin HbA1c, and glucose in blood plasma is measured. Triglycerides in blood plasma was measured every two weeks, starting one week before the lactisole treatment.

TABLE 1
average triglycerides level in blood plasma
of treated and non treated animals

| Duration of | Weeks with | Triglycerides in blood plasma | | |
|---|---|---|---|---|
| high carb | respect to | Lactisole- | Obese | Lean |
| feeding | lactisole | group | controls | controls |
| (weeks) | treatment | (N = 11) | (N = 11) | (N = 12) |
| 0 | −4 | 2.75 | 2.82 | 1.50 |
| 3 | −1 | 2.24 | 2.03 | 1.68 |
| 5 | 1 | 1.82 | 2.86 | 1.70 |
| 7 | 3 | 1.85 | 2.63 | 1.48 |
| 9 | 5 | 1.57 | 2.31 | 1.54 |
| 11 | 7 | 1.52 | 3.01 | 1.51 |

### Difference in Plasma Tryglycerides as Indicator of Metabolic Syndrome

Elevated serum triglycerides are generally accepted as indicator for the presence of a metabolic syndrome in patients with type 2 diabetes (Kompoti et al., 2006). Patients with the metabolic syndrome are at increased risk of coronary heart diseases related to plaque buildups in artery walls. Moreover, high serum triglycerides are significantly correlated to waist circumference in the white population (Lee et al., 2006) and are strongly associated with obesity. Lowering the triglyceride lever should be an aim to limit the cardiovascular risk in obese and/or diabetic patients.

Obese animals were treated for 7 weeks with lactisole (70 mg, two times a day, before feeding). Control obese and lean animals received placebo pills. Whereas the serum triglycerides level in the obese control remains higher than in the lean control, and is even increasing, the serum triglyceride level in the treated obese animals is decreasing to the level of the lean control (Table 1), indicating that lactisole is efficient in treating the primary indicator of the metabolic syndrome.

### Example 4

### Known Anti-Diabetic Compounds do Interact with the Sweet Taste Receptor T1R2-T1R3

Several peroxisome proliferators-activated receptor (PPAR) antagonists are currently being tested in clinical trials as drugs for the treatment of type 2 diabetes mellitus and obesity. However, most of these compounds show a striking structural resemblance with sweet taste inhibitors, and may act not only as PPAR antagonist, but their activity may be based as well, if not predominantly upon their effect on the taste receptor. The effect of naveglitazar [2(S)-methoxy-3-[4-[3-(4-phenoxyphenoxy)propoxy]phenyl]propionic acid], tesaglitazar [(S)-2-ethoxy-3-[4-[2-(4-methanesulfonyloxyphenyl)ethoxy]phenyl]propanoic acid] and LY518674 [2-methyl-2-[4-[3-1-(4-methylbenzyl)-5-oxo-4,5-dihydro-1H-1,2,4-triazol-3-yl]propyl]phenoxy]propionic acid on the signaling of the sweet taste receptor complex is tested in vivo by comparing wild type mice on a low carbohydrate and a high carbohydrate diet, both with and without a suitable amount of PPAR antagonist. SGLT1 expression is measured. The same test is carried out with the T1R3 and α-gustducin knock out mice, proving that the difference in SGLT1 expression is due to the sensing of the PPAR antagonist by the sweet taste receptor.

### REFERENCES

Bellisle, F., Altenburg de Assis, M. A., Fieux, B., Preziosi, P., Guy-Grand, B and Hercberg, S. (2001). Use of ‘light’ foods and drinks in French adults: biological, anthropometric and nutritional correlates. J Hum Nutr Dietet 14, 191-206.
Damak, S., Rong, M., Yasumatsu, K., Kokrashvili, Z., Varadarajan, V., Zou, S., Jiang, P., Ninomiya, Y. and Margolskee, R. F. (2003). Detection of sweet and umami taste in the absence of taste receptor T1R3. Science, 301, 850-853.
Dyer, J., Hosie, K. B. and Shirazi-Beechey, S. P. (1997). Nutrient regulation of human intestinal sugar transporter (SGLT1) expression. Gut, 41, 56-59.
Dyer, J., Vayro, S., King, T. P. and Shirazi-Beechey, S. P. (2003). Glucose sensing in the intestinal epithelium. Eur. J. Biochem. 270, 1-12.
Ferraris, R. P. and Diamond, J. M. (1989). Specific regulation of intestinal nutrient transporters by their dietary substrates. Annu. Rev. Physiol. 51, 125-141.
Goldschmidt, L. A. (2000). Acute and subchronic toxicity of Sucralose. Food Chem Tox 38, S53-S69.
Hill, R. E. and Wood, C. M (1986) 90-day oral toxicity study in the rat ORP-178. unpublished report, submitted to WHO.
Kompoti, M., Mariolis, A., Alevisos, A., Kyrazis, I., Protopsaltis, I., Dimou, E., Lentzas, I., Levisianou, D., Gova, A. and Melidonis, A. (2006) Elevated serum triglycerides is the strongest single indicator for the presence of metabolic syndrome in patients with type 2 diabetes. Cardiovascular Diabetology 5: 21.
Lee, S., Bacha, F. and Arslanian, S. A. (2006) Waist circumference, blood pressure and lipid components of the metabolic syndrome. J. Pediatr. 149, 809-816.
Lescale-Matys, L., Dyer, J., Scott, D., Freeman, T. C., Wright, E. M. and Shirazi-Beechey, S. P. (1993). Regulation of the ovine intestinal Na+/glucose co-transporter (SGLT1) is dissociated from mRNA abundance. Biochem. J. 91, 435-440.
Shirazi-Beechey, S P (1996). Intestinal sodium-dependent D-glucose co-transporter: dietary regulation Proc. Nutr. Soc. 55, 167-178.
Shirazi-Beechey S P, Davies A G, Tebbutt K, Dyer J, Ellis A, Taylor C J, et. al. (1990). Preparation and properties of brush-border membrane vesicles from human small intestine. Gastroenterology, 98, 676-685.
Solberg, D H and Diamond, J M (1987). Comparison of different dietary sugars as inducers of intestinal sugar transporters. Am. J. Physiol. 252, G574-G584.
Winnig, M., Bufe, B. and Meyerhof, W. (2005). Valine 738 and lysine 735 in the fifth transmemebrane domain of rTaslr3 mediate insensitivity towards lactisole of the rat sweet taste receptor. BMC Neuroscience 6, 22.
Wong, G. T., Gannon, K. S. and Margolskee, R. F. (1996). Transduction of bitter and sweet taste by gustducin. Nature, 381, 737-738.

1. The use of a compound inhibiting sweet taste for the preparation of a medicament to treat or prevent obesity and/or to treat or prevent diabetes and/or to lose or control body weight.

2. The use of a compound according to claim 1, whereby said compound has the structure X^{+ -}OOC—(CO)ₘ (CHR)ₙ—(O)_{q}-Φ(R′)_{P}, wherein m represents 0 or 1 and when m is 0, n is 1, 2 or 3 and p represent 1, 2, 3 or 4 and when m is 1, n is 1 or 2 and p is 0, 1, 2, 3 or 4; q represents 0 or 1; R represents H or lower alkyl; R′ represents a lower alkoxy group, a phenoxy group a lower alkyl group or a trifluoromethyl group, or two R′ substituents taken together represent an aliphatic chain linked to the phenyl group (Φ) at two positions, either directly of via an oxa group, or one R′ substituent represent a hydroxyl group while at least one other R′ substituent represents an alkoxy group; X⁺ represents a physiological acceptable cation.

3. The use of a compound according to claim 2, whereby said compound is selected from the group consisting of a propanoic acid derivative, a propionic acid derivative, a methylpropionic acid derivative, a dimethylpropionic acid derivative or an acceptable salt thereof.

4. The use of a compound according to claim 2, whereby said compound is 2-(4-methoxyphenoxy) propanoic acid.

5. The use of a compound according to claim 1, whereby said compound is encapsulated to avoid inactivation of the sweet taste receptor in the mouth.

6. The use of a compound according to claim 1, whereby said compound is encapsulated to be delivered in the intestine.

7. The use of the sweet taste receptor or one of its receptor subunits to screen compounds useful to treat diabetes and/or obesitas.
